(19) United States
(12) Patent Application Publication
(10) Pub. No.: US 2009/0216120 A1
(43) Pub. Date: Aug. 27, 2009

(54) Method for Measuring Hydration of Soft Tissue

(76) Inventor: Raffaella Righetti, Houston, TX (US)

Correspondence Address: BAKER BOTTS L.L.P., 2001 ROSS AVENUE, SUITE 600, DALLAS, TX, 75201-2980, US

(21) Appl. No.: 12/371,357

(22) Filed: Feb. 13, 2009

Related U.S. Application Data
(60) Provisional application No. 61/029,513, filed on Feb. 18, 2008.

(51) Int. Cl. A61B 8/00

(52) U.S. Cl. 600/438

(57) ABSTRACT

The disclosure relates to a method of measuring tissue hydration or a bodily fluid such a lymph or interstitial fluid. In the method, at least one elastographic parameter of a tissue or bodily fluid may be measured to obtain an elastographic parameter measurement that correlates with a mechanical, pathological, physiological or functional property of the tissue or bodily fluid. The elastographic parameter measurement may be correlated with a tissue hydration value or bodily fluid value, including quantitative tissue hydration or bodily fluid values. Multiple measurements taken over time may be used to detect changes in tissue hydration, amounts of bodily fluid, bodily fluid movement, or rate of clearance of a bodily fluid. The disclosure also relates to a device containing a ultrasound transducer, circuitry able to perform methods of the disclosure, and a display able to display tissue hydration or bodily fluid information.

### PRIORITY CLAIM

The present application claims priority under 35 U.S.C. §119 to previously filed U.S. Provisional Patent Application Ser. No. 61/029,513 filed Feb. 18, 2008, which is incorporated by reference herein.

### TECHNICAL FIELD

Embodiments of the current invention relate to method of measuring tissue hydration or bodily fluid movement or rate of clearance using ultrasound-based techniques. Measurements may be specific for a particular tissue, such as a soft tissue, or for a particular bodily fluid. Measurements may be quantitative in that they may involve generation or comparison of numerical values. Soft tissue hydration may be measured to for a variety of reasons, including to determine the effects of exercise on an athlete or the medical state of a patient. In addition to tissue hydration, embodiments of the current invention may be used to measure also other tissue fluids such as lymph or interstitial fluid. Embodiments of the invention also relate to sensors, including portable and hand-held devices, able to perform methods of measuring soft tissue hydration or other bodily fluids.

### BACKGROUND

Tissue hydration assessment techniques usually refer to methods that can be used to assess tissue water content. Tissue hydration assessment techniques may be used in a number of circumstances, including detection of dehydration. Dehydration occurs when excessive water is lost from the body. There are a number of factors that may cause dehydration, such as diseases of the gastrointestinal tract, excessive urination, and excessive sweating, for example due to exercise. When water loss becomes severe, dehydration may be life-threatening. The physiological and mechanical properties of tissues are highly dependent on the water content and water behavior within the tissue. Tissue water content and water mobility may be important diagnostic parameters. Despite their importance, it is difficult to obtain information about tissue water content and water mobility. Knowledge of this information may be useful in a variety of pathological and physiological conditions. In particular, knowledge of the hydration of a particular tissue as opposed to the entire body or large portions of the body may be useful.

Similarly, knowledge about bodily fluid, such as their amounts or movement or rate of clearance, may be very useful diagnostic parameters for a variety of medical conditions, but such knowledge is also hard to obtain, particularly for more diffuse fluids not necessarily contained in an organ, such as lymph or interstitial fluids.

One primary reason for the lack of information regarding tissue hydration or bodily fluids may be the lack of non-invasive methods to accurately and quantitatively measure tissue hydration or properties relating to bodily fluids. Currently, the most common method of measuring tissue hydration in field conditions is body weighing. Body weighing is a fast method that may provide evidence of occurring dehydration. However, it provides on a global measurement of hydration of the entire body. Body weighing provides no information about where water loss has occurred in the body. Additionally, body weighing is not a reliable way to assess rehydration because intake of large amounts of water may rapidly increase body weight, but it may take several hours to days for that water to make its way properly into the tissues.

Another method of assessing hydration focuses on the skin only. Optical coherence tomography and nuclear magnetic resonance imaging have been used to assess tissue hydration. These measurement techniques have several drawbacks. First, they do not directly assess mechanical properties of the skin. Second optical coherence tomography can only image tissues a few millimeters deep, so imaging of the dermis, subcutaneous tissue and muscle tissues is not possible. Nuclear magnetic resonance imaging requires cumbersome, non-portable, and highly specialized equipment (for example the equipment may be dangerous if used near metal objects). Additionally, this equipment may only be used by trained experts.

Other tests for hydration, such as blood tests and bioelectrical impedance measurements, are invasive and often impractical outside of a clinical setting. For some bodily fluid properties, there are no currently available tests, particularly outside of a clinical setting.

### SUMMARY

Embodiments of the current invention may provide a non-invasive and quantitative method for estimating and monitoring the state of hydration of soft tissues or properties relating to bodily fluids. These methods include the use of ultrasound-based techniques. Ultrasound methods may provide one or more of many benefits as compared to the techniques described in the background, including, but not limited to:
Ultrasound methods may focus on one body tissue, allowing the hydration status of that tissue to be specifically studied.
Ultrasound methods may detect rehydration, not mere presence of water in the body that has not entered important tissues.
Ultrasound methods may not be as sensitive to superficial factors such as skin temperature or blood flow as some of the current hydration assessment techniques are.
Ultrasound methods may be used to assess bodily fluid content and mobility such as that of lymph and interstitial fluid.
Ultrasound methods may be safer, more accessible, and/or less expensive than optical coherence tomography and nuclear magnetic resonance imaging.
Ultrasound methods may require less trained technicians to use.
Ultrasound methods may be used in portable devices, allowing them to be more useful on the sports field, in field medicine, military medicine, or in work environments where environmental exposure or physical work may cause dehydration.

One embodiment of the current invention relates to a method of calculating tissue hydration by measuring at least one elastographic parameter of a tissue or bodily fluid to obtain a first elastographic parameter measurement which reflects a mechanical, physiological, pathological, or functional property of the tissue or bodily fluid, then correlating the first elastographic parameter measurement with at least one tissue hydration value or bodily fluid value. The tissue hydration value as well as the elastographic parameter measurements may be quantitative.

Another embodiment of the current invention relates to a method of determining a change in tissue hydration or bodily fluid by measuring at least a first elastographic parameter of a tissue or bodily fluid to obtain a first elastographic parameter measurement which reflects a mechanical, physiological, pathological, or functional property of the tissue or bodily fluid, correlating the first elastographic parameter measurement with a first tissue hydration value or first bodily fluid value, measuring at least a second elastographic parameter of the tissue or bodily fluid to obtain a second elastographic parameter measurement which reflects a mechanical, physiological, pathological, or functional property of the tissue or bodily fluid, correlating the second elastographic parameter measurement with a second tissue hydration value or second bodily fluid value, and then performing a calculation using on the first elastographic parameter and the second elastographic parameter to determine the change in tissue hydration of the tissue or amount or movement of the bodily fluid. The tissue hydration measurements and the elastographic parameter measurements may be quantitative. Further, the first elastographic parameter and the second elastographic parameter may be same type of parameter measured at different times, or they may be different types of elastographic parameters.

Another embodiment of the current invention relates to a method of determining a change in tissue hydration or bodily fluid by measuring at least a first elastographic parameter of a tissue or bodily fluid to obtain a first elastographic parameter measurement which reflects a mechanical, physiological, pathological, or functional property of the tissue or bodily fluid, measuring at least a second elastographic parameter of the tissue or bodily fluid to obtain a second elastographic parameter measurement which reflects a mechanical, physiological, pathological, or functional property of the tissue or bodily fluid, performing a calculation using on the first elastographic parameter and the second elastographic parameter, and then correlating the result of the calculation using on the first elastographic parameter and the second elastographic parameter with a tissue hydration value or bodily fluid value. The tissue hydration measurements and the elastographic parameter measurements may be quantitative. Further, the first elastographic parameter and the second elastographic parameter may be same type of parameter measured at different times, or they may be different types of elastographic parameters.

Still another embodiment of the invention relates to a tissue hydration or bodily fluid sensor including a ultrasound transducer, circuitry able to carry out either of the above methods or any other method of the present invention, and a display able to display the tissue hydration value or bodily fluid value, the change in tissue hydration of the tissue or a change in amount or movement if a bodily fluid, and/or any other information determined using a method of the current invention.

Various embodiments of the present invention may be better understood though the following detailed description.

### DETAILED DESCRIPTION

Embodiments of the current invention may provide a non-invasive method for estimating and monitoring the state of hydration of soft tissues, including dehydration and rehydration and/or properties of bodily fluids such as amount and/or movement of these fluids and/or rate of clearance. Some methods may provide quantitative results. As used in the present specification, “quantitative” shall mean “relating to the generation or comparison of numerical values.”

Some methods of the current invention may use an ultrasound technique known as elastography. Elastography creates images of the strains or other tissue parameters that a soft tissue experiences when a compressive force is applied. In particular embodiments, the time-dependent behavior of a tissue may be related to its state of hydration.

Similarly, the responses to compressive forces may be used to determine the amount of a bodily fluid and time-dependent behavior may be used to determine movement or rate of clearance of bodily fluids. Although specific embodiments discussed herein may relate to tissue fluids, these embodiments may be used in relation to bodily fluid parameters as well.

Methods of the present invention, in particular embodiments, may a) be non-invasive; b) provide a quantitative measurement, such as a precise estimate of the fluid flow time constant in a tissue or the fluid flow constant for a bodily fluid; c) be implemented with a portable device; and/or d) provide local measurements of tissue hydration or properties of bodily fluids.

Methods of the present invention, unlike conventional ultrasound methods, may not require the use of high frequency transducers, which limit the penetration depth and area of investigation to a few millimeters. Because a goal of some methods of the present invention is to measure the mechanical properties of tissue and not sonographic properties of the skin, the methods may be implemented using diagnostic low frequency transducers, which allow tissue penetration of several centimeters. For example, the ultrasound frequency in some embodiments may be as low as 0.1 MHz. In general, diagnostic frequencies in some embodiments of the invention may be between 0.1 MHz and 50 MHz. Thus, these methods may be used to measure tissue hydration in the skin, dermis and muscle tissues or bodily fluids in these tissues. Further, in certain embodiments, the methods may be used to determine hydration, dehydration, rehydration, fluid content, fluid flow movement, fluid flow rate, or fluid flow pattern. They may measure changes in these states over time. Thus, these methods may be used to measure tissue hydration and changes in tissue hydration as well as the presence, location, or movement or rate of clearance of bodily fluids such as lymph or interstitial fluid.

In a particular embodiment of the current invention, a diagnostic ultrasound transducer may be placed in contact with a tissue of interest or the location of the bodily fluid of interest. Then, a small compression may be applied to the tissue or location. The compression may be a force or a displacement. It may be an external compression or an internal compression. Ultrasound data is acquired before and after compression. The acquired ultrasound data is used to determine the local strain or strains that the tissue experiences due to the compression. Local strain time constants are related to the local viscoelastic and poroelastic properties of the tissue. These properties depend on the hydration state of the tissues. Thus, the measured strain time constants may be correlated to a quantitative amount of hydration. Similarly, the acquired ultrasound data can be used to determine the local displacement or displacements that the tissue experiences due to the compression. Local displacement time constants are related to the local viscoelastic and poroelastic properties of the tissue. Thus, the measured displacement time constants may be correlated to a quantitative amount of hydration.

Methods of the current invention may involve the measurement of various tissue parameters using elastography techniques. These tissue parameters may include a mechanical, physiological, pathological, or functional property that may be measured using elastographic techniques. For example, axial strain may be measured. Axial strain is the estimated strain tensor components along the ultrasonic beam axis. Axial strain time constants may also be measured. These time constants relate to the time constant of the temporal behavior of each pixel or data point in a poroelastogram (a time-dependent image) of the axial strain. Lateral displacement may also be measured. Lateral strain is the estimated tensor strain components orthogonal to the ultrasonic beam axis within the scanning plane. Lateral strain time constants may be measured in a manner similar to axial strain time constants. Lateral-to-axial displacement ratio and lateral-to-axial strain ratio may be measured. Lateral-to-axial displacement ratio and lateral-to-axial strain ratio time constants may be measured in a manner similar to axial strain time constants.

In addition to these measurements, the following elastography measurements, alone or in combinations, may be used to determine tissue hydration: axial strain slope parameters, lateral strain slope parameters, axial displacements, axial displacement time constants, axial displacement slope parameters, elevational strains, elevational strain time constants, elevational strain slope parameters, shear strains, shear strain time constants, shear strain slope parameters, strain ratios, strain ratios time constants, strain ratios slope parameters, lateral displacements, lateral displacement time constants, lateral displacement slope parameters, elevational displacements, elevational displacement time constants, elevational displacement slope parameters, displacement ratios, displacement ratios time constants, displacement ratios slope parameters, axial strain rate, lateral strain rate, elevational strain rate, shear strain rate, strain ratio rate, axial displacement rate, lateral displacement rate, elevational displacement rate, displacement ratio rate, cross-correlation, cross-correlation ratio, cross-correlation time constants, cross-correlation slope parameters, any polynomial comprising at least 1 coefficient defining a functional relationship between strains measured at 2 different times, any polynomial comprising at least 1 coefficient defining a functional relationship between displacements measured at 2 different times, any polynomial comprising at least 1 coefficient defining a functional relationship between cross-correlation parameters measured at 2 different times, any polynomial comprising at least 1 coefficient defining a functional relationship between strain ratios measured at 2 different times, any polynomial comprising at least 1 coefficient defining a functional relationship between displacement ratios measured at 2 different times, any polynomial comprising at least 1 coefficient defining a functional relationship between strains and time constants measured at 2 different times, any polynomial comprising at least 1 coefficient defining a functional relationship between strains and strain time constants measured at 2 different times, any polynomial comprising at least 1 coefficient defining a functional relationship between displacements and displacement time constants measured at 2 different times, any polynomial comprising at least 1 coefficient defining a functional relationship between strain ratios and strain ratio time constants measured at 2 different times, any polynomial comprising at least 1 coefficient defining a functional relationship between cross-correlation and cross-correlation time constants measured at 2 different times, any polynomial comprising at least 1 coefficient defining a functional relationship between strains and displacement time constants measured at 2 different times, any polynomial comprising at least 1 coefficient defining a functional relationship between strains and strain ratio time constants measured at 2 different times, any polynomial comprising at least 1 coefficient defining a functional relationship between strains and cross-correlation time constants measured at 2 different times, any polynomial comprising at least 1 coefficient defining a functional relationship between any combination of elastographic parameters measured at 2 different times, and any combinations thereof. Further, different curve fitting parameters may be used to determine the decay or the increment of strains or displacement or cross-correlation values or any combination of the above in the tissue. In addition to these measurements, the normalized values, the mean values or the average values of the above elastographic measurements, alone or in combinations, may be used to determine tissue hydration. In one specific embodiment, the difference between elastographic measurements or the difference between elastographic measurements divided by the first measurement or the difference between elastographic measurements divided by the second measurement may be used to calculate tissue hydration changes.

In particular embodiments, the elastographic parameters measured and how they are used may vary. For example, a single elastographic parameter may be measured and then correlated with tissue hydration or a bodily fluid value. The single parameter may be measured once or multiple times. In another example, two parameters may be measured or two measurements may be taken of the same parameter. These measurements may be used to determine a change in tissue hydration or a bodily fluid value. They may also independently be correlated with a tissue hydration or bodily fluid value or may be combined to correlate with such a value. In other embodiments with two measurements or two parameters measured a calculation may be performed using the resulting measurements and the result of the calculation may be correlated with a hydration value or bodily fluid value.

All of the above measurements may be taken or treated quantitatively in some embodiments. In other embodiments they may be non-quantitative, or may not be subject to further quantitative treatment.

Tissue parameters may be correlated with hydration via a variety of techniques. For example, an equation may be developed to calculate hydration based on one or more parameters. Alternatively, a table correlating a parameter value with a particular hydration value may be used. Interpolation may be employed in this technique. In either method, the measured parameter may be correlated with a quantitative tissue hydration value.

Methods of the present invention may use direct contact of the ultrasound transducer and the body or a coupling material may be used between the transducer and the body.

Methods of the present invention may use some techniques also used in previous ultrasound methods. For example, ultrasound has been used previously to image the echo properties of the upper dermis to provide a qualitative estimate of general skin condition. These techniques do not allow visualization of tissue mechanical properties or any imaging of water mobility within the tissue. Therefore, these techniques are not suitable to quantitatively determine water content in the dermis or other tissues. However, elements of these techniques may be useful in embodiments of the present invention. Accordingly, Gniadecka, M. and Quistorff, B., “Assessment of dermal water by high-frequency ultrasound; comparative studies with nuclear magnetic resonance.” Brit. J. Dermat., 135:218-224 (1996) and Rallan D, Harland C C, “Ultrasound in dermatology—basic principles and applications. Review.” Exp. Dermat. 28:632-638 (2003) are both incorporated by reference herein.

In particular, methods of the present invention may be similar to method previously used to detect fluid content and fluid mobility in poroelastic phantoms. Such a method is described in Righetti, R. et al., “The feasibility for estimating and imaging the mechanical behavior of poroelastic materials using axial strain elastography.” Physics in Medicine and Biology, 52(11): 3241-3259 (2007), incorporated by reference herein. These methods are adapted in the present invention to detect hydration state of actual living tissues.

Methods used to monitor the time-dependent behavior of edematous tissues in vivo may also be adapted for use in the current invention. Such methods are described in Righetti, R. et al., “The feasibility of using poroelastographic techniques for distinguishing between normal and lymphedematous tissues in vivo.” Physics in Medicine and Biology, 52: 6525-6541 (2007), incorporated by reference herein.

Additional useful techniques may be found in U.S. Pat. No. 5,474,070 and US 2008/0009721, both incorporated by reference herein.

Some methods of the invention may employ spatial and temporal averaging techniques to reduce noise and improve the signal-to-noise ratio.

Other methods may address high variability in some hydrated tissues by measuring the relative differences in the state of hydration of a tissue before and after the tissue has been exposed to conditions that may cause dehydration or conditions that may cause rehydration. For example, a measurement may be taken before and after physical exertion to detect dehydration caused by the exertion. Further, variability may be lessened if multiple parameters are used to determine tissue hydration. For example, axial strain values may be used in addition to axial strain time constant in some methods.

Further, the measurement sensitivity for measurement parameters may be adjusted to obtain desired accuracy of hydration measurements, or parameters may be selected based on the level of sensitivity that may be obtained. For example, elastography techniques in vivo have been shown to have a general sensitivity of below 0.1 strain and these are able to detect axial strain changes that are significantly lower than the changes (typically body loss of greater than 2%) that occur between hydrated and dehydrated tissues.

The current invention additionally includes sensors able to implement methods of the present invention. These sensors may include an ultrasound transducer, circuitry to receive and interpret the ultrasound signal according to a method of the current invention, and a display or other device to communicate quantitative information about tissue hydration or bodily fluids.

The sensor may be able to implement more than one method of the present invention and may allow the user to select an appropriate method. It may also select the method itself based on various parameters input by the user or detected by the device.

The display in the sensor may provide a numeric value of tissue hydration, or it may include a simpler indicator that the value is within a certain range or even merely indicate that the tissue in a certain state of hydration, such as dehydration. Other information able to be detected using methods of the invention may also be displayed. In certain embodiments, the display may be replaced by or supplemented with an auditory indicator.

The sensor may include memory storage circuitry, which may be integral or detachable, to allow multiple measurements, for example multiple measurements from the same person, to be stored. These measurements may be compared to detect changes in hydration. Information about changes in hydration may also be provided on the display. Although some sensors without memory storage circuitry may provide information about fluid movement or bodily fluids, sensors with memory storage circuitry may use this circuitry to store information from multiple measurements and thus perform more complicated calculations regarding fluid movement, such as bodily fluid movement.

Although only exemplary embodiments of the invention are specifically described above, it will be appreciated that modifications and variations of these examples are possible without departing from the spirit and intended scope of the invention. For example, as discussed earlier, methods and devices for use in determining tissue hydration may be used with or without modification by one of ordinary skill in the art to make measurements pertaining to bodily fluids such as interstitial fluid or lymph. For example, such methods or devices may take advantages in differences in time constants for bodily fluids as compared to one another or tissue hydration measurements.

1. A method of calculating tissue hydration comprising:
measuring at least one elastographic parameter of a tissue or bodily fluid to obtain a first elastographic parameter measurement which reflects a mechanical, physiological, pathological, or functional property of the tissue or bodily fluid; and
correlating the first elastographic parameter measurement with at least one tissue hydration value or bodily fluid value.

2. The method according to claim 1, wherein correlating comprises using an equation to correlate the elastographic parameter measurement with the tissue hydration value or bodily fluid value.

3. The method according to claim 1, wherein correlating comprises using a table to correlate the elastographic parameter measurement with the tissue hydration value or bodily fluid value.

4. The method according to claim 1, wherein the elastographic parameter is selected from the group consisting of: axial strain, axial strain time constants, lateral strain, lateral strain time constants, axial strain slope parameters, lateral strain slope parameters, axial displacements, axial displacement time constants, axial displacement slope parameters, elevational strains, elevational strain time constants, elevational strain slope parameters, shear strains, shear strain time constants, shear strain slope parameters, strain ratios, strain ratios time constants, strain ratio slope parameters, lateral displacements, lateral displacement time constants, lateral displacement slope parameters, elevational displacements, elevational displacement time constants, elevational displacement slope parameters, displacement ratios, displacement ratios time constants, displacement ratios slope parameters, axial strain rate, lateral strain rate, elevational strain rate, shear strain rate, strain ratio rate, axial displacement rate, lateral displacement rate, elevational displacement rate, displacement ratio rate, cross-correlation, cross-correlation ratio, cross-correlation time constants, cross-correlation slope parameters, any polynomial comprising at least 1 coefficient defining a functional relationship between strains measured at 2 different times, any polynomial comprising at least 1 coefficient defining a functional relationship between displacements measured at 2 different times, any polynomial comprising at least 1 coefficient defining a functional relationship between cross-correlation parameters measured at 2 different times, any polynomial comprising at least 1 coefficient defining a functional relationship between strain ratios measured at 2 different times, any polynomial comprising at least 1 coefficient defining a functional relationship between displacement ratios measured at 2 different times, any polynomial comprising at least 1 coefficient defining a functional relationship between any combination of strains measured at 2 different times, any polynomial comprising at least 1 coefficient defining a functional relationship between any combination of displacements measured at 2 different times, any polynomial comprising at least 1 coefficient defining a functional relationship between any combination of elastographic parameters measured at 2 different times, and any combinations thereof.

5. The method according to claim 1, further comprising measuring at least a second elastographic parameter of a tissue or bodily fluid to obtain a second elastographic parameter measurement with reflects a mechanical, physiological, pathological, or functional property of the tissue or bodily fluid; and
correlating the second elastographic parameter measurement or any combination of the first and second elastographic parameter measurements with the at least one tissue hydration value or bodily fluid value.

6. The method according to claim 1, further comprising measuring at least a second elastographic parameter of a tissue or bodily fluid to obtain a second elastographic parameter measurement with reflects a mechanical, physiological, pathological, or functional property of the tissue or bodily fluid; and
performing a calculation using on the first elastographic parameter measurement and the second elastographic parameter measurement; and correlating the result of the calculation with a hydration value or bodily fluid value.

7. The method according to claim 1, wherein measuring an elastographic parameter comprises contacting an ultrasound transducer producing a diagnostic frequency with the tissue.

8. The method according to claim 1, further comprising applying a spatial or temporal averaging technique to the elastographic parameter measurement.

9. The method according to claim 1, wherein the tissue comprises a skin, dermal or muscle tissue.

10. The method according to claim 1, wherein the bodily fluid comprises lymph or interstitial fluid.

11. The method according to claim 1, wherein the tissue hydration value or bodily fluid value comprises a quantitative tissue hydration value or bodily fluid value.

12. A method of determining a change in tissue hydration or bodily fluid comprising:
measuring at least a first elastographic parameter of a tissue or bodily fluid to obtain a first elastographic parameter measurement which reflects a mechanical, physiological, pathological, or functional property of the tissue or bodily fluid;
correlating the first elastographic parameter measurement with a first tissue hydration value or first bodily fluid value;
measuring at least a second elastographic parameter of the tissue or bodily fluid to obtain a second elastographic parameter measurement which reflects a mechanical, physiological, pathological, or functional property of the tissue or bodily fluid;
correlating the second elastographic parameter measurement with a second tissue hydration value or second bodily fluid value; and
performing a calculation using on the first elastographic parameter and the second elastographic parameter to determine the change in tissue hydration of the tissue or amount, movement, or rate of clearance of the bodily fluid.

13. The method according to claim 12, wherein the first elastographic parameter and the second elastographic parameter comprise the same type of parameter measured at different times.

14. The method according to claim 12, wherein the first elastographic parameter and the second elastographic parameter comprise different types of elastographic parameters.

15. The method according to claim 12, wherein one or both correlating steps comprise using an equation to correlate the elastographic parameter measurement with the tissue hydration value or bodily fluid value.

16. The method according to claim 12, wherein one or both correlating steps comprise using a table to correlate the elastographic parameter measurement with the tissue hydration value or bodily fluid value.

17. The method according to claim 12, wherein one or both of the first and second elastographic parameters is selected from the group consisting of: axial strain, axial strain time constants, lateral strain, lateral strain time constants, axial strain slope parameters, lateral strain slope parameters, axial displacements, axial displacement time constants, axial displacement slope parameters, elevational strains, elevational strain time constants, elevational strain slope parameters, shear strains, shear strain time constants, shear strain slope parameters, strain ratios, strain ratios time constants, strain ratio slop parameters lateral displacements, lateral displacement time constants, lateral displacement slope parameters, elevational displacements, elevational displacement time constants, elevational displacement slope parameters, displacement ratios, displacement ratios time constants, displacement ratios slope parameters, axial strain rate, lateral strain rate, elevational strain rate, shear strain rate, strain ratio rate, axial displacement rate, lateral displacement rate, elevational displacement rate, displacement ratio rate, cross-correlation, cross-correlation ratio, cross-correlation time constants, cross-correlation slope parameters, any polynomial comprising at least 1 coefficient defining a functional relationship between strains measured at 2 different times, any polynomial comprising at least 1 coefficient defining a functional relationship between displacements measured at 2 different times, any polynomial comprising at least 1 coefficient defining a functional relationship between cross-correlation parameters measured at 2 different times, any polynomial comprising at least 1 coefficient defining a functional relationship between strain ratios measured at 2 different times, any polynomial comprising at least 1 coefficient defining a functional relationship between displacement ratios measured at 2 different times, any polynomial comprising at least 1 coefficient defining a functional relationship between any combination of strains measured at 2 different times, any polynomial comprising at least 1 coefficient defining a functional relationship between any combination of displacements measured at 2 different times, any polynomial comprising at least 1 coefficient defining a functional relationship between any combination of elastographic parameters measured at 2 different times, and any combinations thereof.

18. The method according to claim 12, wherein measuring both the first and second elastographic parameters comprises contacting an ultrasound transducer producing a diagnostic frequency with the tissue or a tissue surrounding or containing the bodily fluid.

19. The method according to claim 12, further comprising applying a spatial or temporal averaging technique to one or both of the first and second elastographic parameter measurements.

20. The method according to claim 12, wherein the tissue comprises a skin, dermal or muscle tissue.

21. The method according to claim 12, wherein the bodily fluid comprises lymph or interstitial fluid.

22. The method according to claim 12, wherein the first and second tissue hydration values or bodily fluid values each comprises a quantitative tissue hydration value.

23. A tissue hydration or bodily fluid sensor comprising:
an ultrasound transducer operable to produce a diagnostic ultrasound frequency;
circuitry operable to:
measure at least a first elastographic parameter of a tissue or a bodily fluid to obtain a first elastographic parameter measurement which reflects a mechanical, physiological, pathological, or functional property of the tissue or bodily fluid; and
correlate the elastographic parameter measurement with a first tissue hydration value or first bodily fluid value;
a display able to display the tissue hydration value or bodily fluid value.

24. The sensor of claim 23, further comprising:
a memory device able to store the first tissue hydration value or bodily fluid value;
circuitry able to:
measure at least a second elastographic parameter of the tissue or bodily fluid to obtain a second elastographic parameter measurement which reflects a mechanical, physiological, pathological, or functional property of the tissue or bodily fluid; and
correlate the second elastographic parameter measurement with a second tissue hydration value or second bodily fluid value;
circuitry able to:
perform a calculation using the first tissue elastographic value and the second elastographic value to determine a change in tissue hydration of the tissue or change in amount or movement of the bodily fluid; and
a display able to display the change in tissue hydration of the tissue or change in amount, movement, or rate of clearance of the bodily fluid.

25. The sensor of claim 23, further comprising:
a memory device able to store the first tissue hydration value or bodily fluid value;
circuitry able to:
measure at least a second elastographic parameter of the tissue or bodily fluid to obtain a second elastographic parameter measurement which reflects a mechanical, physiological, pathological, or functional property of the tissue or bodily fluid; and
correlate the second elastographic parameter measurement with a second tissue hydration value or second bodily fluid value;
circuitry able to:
perform a calculation using the first tissue hydration value and the second hydration value to determine a change in tissue hydration of the tissue or change in amount or movement of the bodily fluid; and
a display able to display the change in tissue hydration of the tissue or change in amount, movement, or rate of clearance of the bodily fluid.

26. The sensor of claim 23, further comprising:
a memory device able to store the first tissue hydration value or bodily fluid value;
circuitry able to:
measure at least a second elastographic parameter of the tissue or bodily fluid to obtain a second elastographic parameter measurement which reflects a mechanical, physiological, pathological, or functional property of the tissue or bodily fluid;
circuitry able to:
perform a calculation using the first elastographic parameter and the second elastographic parameter; and
correlate the value resulting from the calculation with the tissue hydration value or bodily fluid value and
a display able to display the tissue hydration of the tissue or amount, movement, or rate of clearance of the bodily fluid.
